(19) United States
(12) Patent Application Publication
(10) Pub. No.: US 2006/0040941 A1
(43) Pub. Date: Feb. 23, 2006

(54) 7-Aminoimidazotriazones

(75) Inventors: Cristina Alonso-Alija, Haan (DE); Heike Gielen-Haertwig, Monheim (DE); Martin Michels, Solingen (DE); Dagmar Karthaus, Solingen (DE); Hilmar Bischoff, Wuppertal (DE); Nils Burkhardt, Velbert (DE); Volker Geiss, Ratingen (DE); Karl-Heinz Schlemmer, Wuppertal (DE); Nigel Cuthbert, Devon (GB); Mary F. Fitzgerald, Oxford (GB); Graham Sturton, Berkshire (GB); Ulrich Niewohner, Wermelskirchen (DE); Maria Niewohner, Wermelskirchen (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 10/513,116

(22) PCT Filed: Apr. 22, 2003
(86) PCT No.: PCT/EP03/04129

§ 371 Date: Jun. 6, 2005

(30) Foreign Application Priority Data
May 1, 2002 (GB) 0209988.5

(51) Int. Cl. A61K 31/53; C07D 487/04

(52) U.S. Cl. 514/243; 544/184

(57) ABSTRACT

The invention relates to novel 7-amino-imidazotriazinones, processes for their preparation and their use in medicaments, esp. for the treatment and/or prophylaxis of inflammatory processes and/or immune diseases.

The invention relates to novel 7-amino-imidazotriazinones, processes for their preparation and their use in medicaments, esp. for the treatment and/or prophylaxis of inflammatory processes and/or immune diseases.

Phosphodiesterases (PDEs) are a family of enzymes responsible for the metabolism of the intracellular second messengers cAMP (cyclic adenosine monophosphate) and cGMP (cyclic guanosine monophosphate). PDE 4, as a cANP specific PDE, catalyses the conversion of cAMP to AMP and is the major if not sole isoform of the phosphodiesterase enzymes present in inflammatory and immune cell types. Inhibition of this enzyme leads to the accumulation of cAMP which, in these cells, leads to the inhibition of a range of pro-inflammatory functions. Uncontrolled production of inflammatory mediators can lead to acute and chronic inflammation, tissue damage, multi-organ failures and to death. Additionally, elevation of phagocyte cAMP leads to inhibition of oxygen radical production. This cell function is more sensitive than others such as aggregation or enzyme release.

It is now recognised that both asthma and COPD (Chronic obstructive pulmonary disease) are chronic inflammatory lung diseases. In the case of asthma the eosinophil is the predominant infiltrating cell. Subsequent release of superoxide radicals as well as damaging cationic proteins from these infiltrating cells are believed to play a role in the progression of the disease and development of airway hyperreactivity.

By contrast, in COPD the neutrophil is the predominant inflammatory cell type found in the lungs of sufferers. The action of mediators and proteases released in the environment of the lung is believed to result in the irreversible airway obstruction seen in COPD. In particular the action of proteases in degrading the lung matrix results in fewer alveoli and is likely to be the major cause of accelerated long term lung function decline seen in this disease.

Treatment with a PDE 4 inhibitor is expected to reduce the inflammatory cell burden in the lung in both of these diseases [M. S. Barnette, “PDE 4 inhibitors in asthma and chronic obstructive pulmonary disease”, in: Progress in Drug Research, Birkihäuser Verlag, Basel, 1999, pp. 193-229; H. J. Dyke and J. G. Montana, “The therapeutic potential of PDE 4 inhibitors”, Exp. Opin. Invest. Drugs 8, 1301-1325 (1999)].

WO 99/24433 and WO 99/67244 describe 2-phenyl-imidazotriazinones as synthetic intermediates for the synthesis of 2-(aminosulfonyl-phenyl)-imidazotriazinones as inhibitors of cGMP-metabolizing phosphodiesterases.

U.S. Pat. No. 4,278,673 discloses 2-aryl-imidazotriazinones with cAMP-phosphodiesterase inhibitory activity for the treatment of i.a. asthma.

The present invention relates to compounds of the general formula (I) 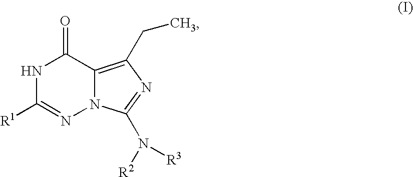
in which
R¹ denotes phenyl or 5- to 6-membered heteroaryl, which can be substituted by 0, 1, 2 or 3 residues independently selected from the group consisting of halogen, (C₁-C₄)-alkyl, (C₁-C₄)-alkoxy, trifluoromethyl, cyano, nitro and trifluoromethoxy,
R² denotes hydrogen, (C₁-C₈)-alkyl or (C₃-C₈)-cycloalkyl, and
R³ denotes (C₁-C₈)-alkyl or (C₃-C₈)-cycloalkyl, or
NR²R³ denotes optionally benzannelated, 4- to 10-membered heterocyclyl,
   which contains at least one nitrogen ring atom,
   which is attached to the imidazotriazinone by a nitrogen ring atom, and
   which can be substituted by 0, 1, 2 or 3 residues independently selected from the group consisting of halogen, (C₁-C₆)-alkyl, (C₁-C₆)-alkoxy, (C₁-C₆)-alkoxycarbonyl, hydroxy, halogen, trifluoromethyl and oxo.

The compounds according to this invention can also be present in the form of their salts, hydrates and/or solvates.

In general, salts with organic or inorganic bases or acids may be mentioned here.

Physiologically acceptable salts are preferred in the context of the present invention.

Physiologically acceptable salts can also be salts of the compounds according to this invention with inorganic or organic acids. Preferred salts are those with inorganic acids such as, for example, hydrochloric acid, hydrobromic acid, phosphoric acid or sulphuric acid, or salts with organic carboxylic or sulphonic acids such as, for example, acetic acid, maleic acid, fumaric acid, malic acid, citric acid, tartaric acid, ethanesulphonic acid, benzenesulphonic acid, toluenesulphonic acid or naphthalenedisulphonic acid. Preferred pyridinium salts are salts in combination with halogen.

The compounds according to this invention can exist in stereoisomeric forms which either behave as image and mirror image (enantiomers), or which do not behave as image and mirror image (diastereomers). The invention relates both to the enantiomers and to the racemates, as well as the pure diastereomer and mixtures thereof. The racemates, like the diastereomers, can be separated into the stereoisomerically uniform constituents according to known methods.

Hydrates of the compounds of the invention are stoichiometric compositions of the compounds with water, such as for example hemi-, mono-, or dihydrates.

Solvates of the compounds of the invention or their salts are stoichiometric compositions of the compounds with solvents.

(C₁-C₆)— and (C₁-C₄)-Alkoxy in general represent straight chain or branched alkoxy residues with 1 to 6 or 1 to 4 carbon atoms, respectively. The following alkoxy residues are mentioned by way of example: methoxy, ethoxy, n-propoxy, isopropoxy, tert.-butoxy, n-pentoxy and n-hexoxy. Alkoxy residues with 1 to 4 carbon atoms are preferred. Alkoxy residues with 1 to 3 carbon atoms are especially preferred.

(C₁-C₆)— and (C₁-C₄)-Alkoxycarbonyl in general represent straight chain or branched alkoxy residues with 1 to 6 or I to 4 carbon atoms, respectively, attached to a carbonyl group. The alkoxy residue is defined as above. Methoxycarbonyl and ethoxycarbonyl are preferred.

(C₁-C₈—, (C₁-C₆)—, and (C₁-C₄)-Alkyl in general represent straight chain or branched alkyl residues with 1 to 8, 1 to 6 or 1 to 4 carbon atoms, respectively. The alkyl residues can be saturated or partially unsaturated, i.e. contain one or more double and/or triple bonds. Saturated alkyl residues are preferred. The following alkyl residues are mentioned by way of example: methyl, ethyl, n-propyl, isopropyl, allyl, propargyl, tert.-butyl, pentyl, hexyl, heptyl, and octyl.

(C₃-C₈)-Cycloalkyl in general represents a cycloalkyl residue with 3 to 8 carbon atoms. The following cycloalkyl residues are mentioned by way of example: cyclopropyl, cyclobutyl, cyclopentyl, cyclopentenyl, cyclohexyl, cycloheptyl, and cyclooctyl. Cyclopentenyl and cyclohexyl are preferred.

Halogen in general represents fluoro, chloro, bromo and iodo. Fluoro, chloro and bromo are preferred. Fluoro and chloro are especially preferred.

4- to 10-membered heterocyclyl in general represents a mono- or polycyclic, heterocyclic residue with 4 to 10 ring atoms. The heterocyclyl residue can contain up to 3, preferentially 1, hetero ring atoms selected from nitrogen, oxygen, sulfur, —SO—, —SO₂—. Nitrogen is preferred. Mono- and bicyclic heterocyclyl residues are preferred. Especially preferred are monocyclic heterocyclyl residues. The heterocyclyl residues can be saturated or partially unsaturated. Saturated heterocyclyl residues are preferred. The following heterocyclyl residues are mentioned by way of example: pyrrolidinyl, pyrrolinyl, piperidinyl, piperazinyl morpholinyl, perhydroazepinyl.

Oxo in general represents a double-bonded oxygen atom.

Unless specified otherwise, when groups in compounds of the invention are optionally substituted, substitution by up to three identical or different residues is generally preferred.

A preferred embodiment of the invention relates to compounds of the general formula (I), in which

R¹ denotes phenyl, which can be substituted by 0, 1, 2 or 3 residues independently selected from the group consisting of fluoro, chloro, methyl, ethyl, trifluoromethyl and cyano, and

R²and R³ have the meaning indicated above.

Especially preferred are compounds of the general formula (I), in which

R¹ denotes phenyl, and

R² and R³ have the meaning indicated above.

Another preferred embodiment of the invention relates to compounds of the general formula (I), in which

R¹ has the meaning indicated above, and

R² and R³ are identical or different and denote (C₁-C₆)-alkyl or (C₃-C₈)-cycloalkyl.

Another preferred embodiment of the invention relates to compounds of the general formula (I), in which

R¹ has the meaning indicated above, and

NR²R³ denotes optionally benzannelated, 4- to 10-membered heterocyclyl,
which contains at least one nitrogen ring atom,
which is attached to the imidazotriazinone by a nitrogen ring atom, and
which is optionally substituted by identical or different residues selected from the group consisting of (C₁-C₄)-alkyl, (C₁-C₄)-alkoxy, and (C₁-C₄)-alkoxy-carbonyl.

Especially preferred are compounds of the general formula (I), in which

R′ has the meaning indicated above, and

NR²R³ denotes optionally benzannelated pyrrolidin-1-yl, morpholin-1-yl, piperidin-1-yl, piperazin-1-yl, 2-aza-bicyclo[3.2.0]heptan-2-yl, 2-aza-bicyclo-[3.2.1]octan-2-yl, which are optionally substituted by one to three identical or different residues selected from the group consisting of (C₁-C₄)-alkyl, (C₁-C₄)-alkoxy, and (C₁-C₄)-alkoxycarbonyl.

Another preferred embodiment of the invention relates to compounds of the general formula (I), in which
R¹ denotes phenyl, which can be substituted by 0, 1, 2 or 3 residues independently selected from the group consisting of fluoro, chloro, methyl, ethyl, trifluoromethyl and cyano, and
R² and R³ are identical or different and denote (C₁-C₆)-alkyl or (C₃-C₈)-cyclo-alkyl, or
NR²R³ denotes optionally benzannelated, 4- to 10-membered heterocyclyl,
   which contains at least one nitrogen ring atom,
   which is attached to the imidazotriazinone by a nitrogen ring atom, and
   which can be substituted by 0, 1, 2 or 3 residues independently selected from the group consisting of (C₁-C₄)-alkyl, (C₁-C₄)-alkoxy, and (C₁-C₄)-alkoxycarbonyl.

The invention furthermore provides a process for preparing the compounds of the general formula (I) according to the invention, characterized in that compounds of the general formula (II) 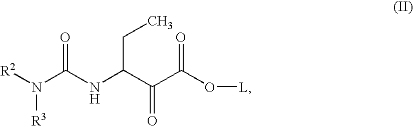
in which

R² and R³ are as defined above and

L represents straight-chain or branched alkyl having up to 4 carbon atoms, are condensed with compounds of the general formula (III)

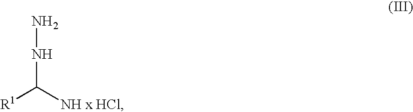

in which

R¹ is as defined above,

preferably using ethanol as a solvent, to the compounds of the general formula (IV)

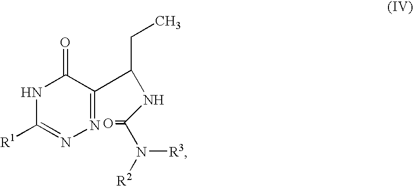

in which

R¹, R² and R³ are as defined above,

which can optionally after isolation be reacted with a dehydrating agent, preferably phosphorous oxytrichloride, to yield the compounds of the general formula (I).

The compounds of the general formula (I) can alternatively be prepared by

[A] condensation of compounds of the general formula (IIa) 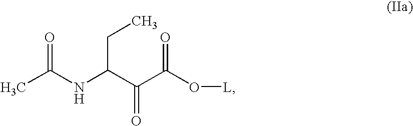
in which
L is as defined above,
with compounds of the general formula (III) to compounds of the general formula (IVa) 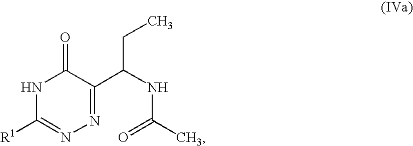
in which
R¹ is as defined above,
preferably using ethanol as a solvent,

[B] followed by hydrolysis of the compounds of the general formula (Iva) to compounds of the general formula (V) 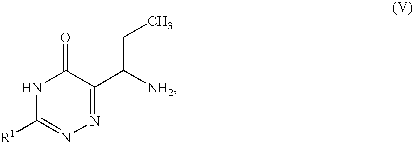
in which
R¹ is as defined above,

[C] and finally by condensation of the compounds of the general formula (V) with compounds of the general formula (VI) 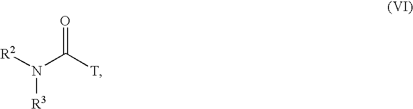
in which
R² and R³ are as defined above, and
T represents a leaving group, preferably chlorine.

The process according to the invention can be illustrated using the following scheme as an example:

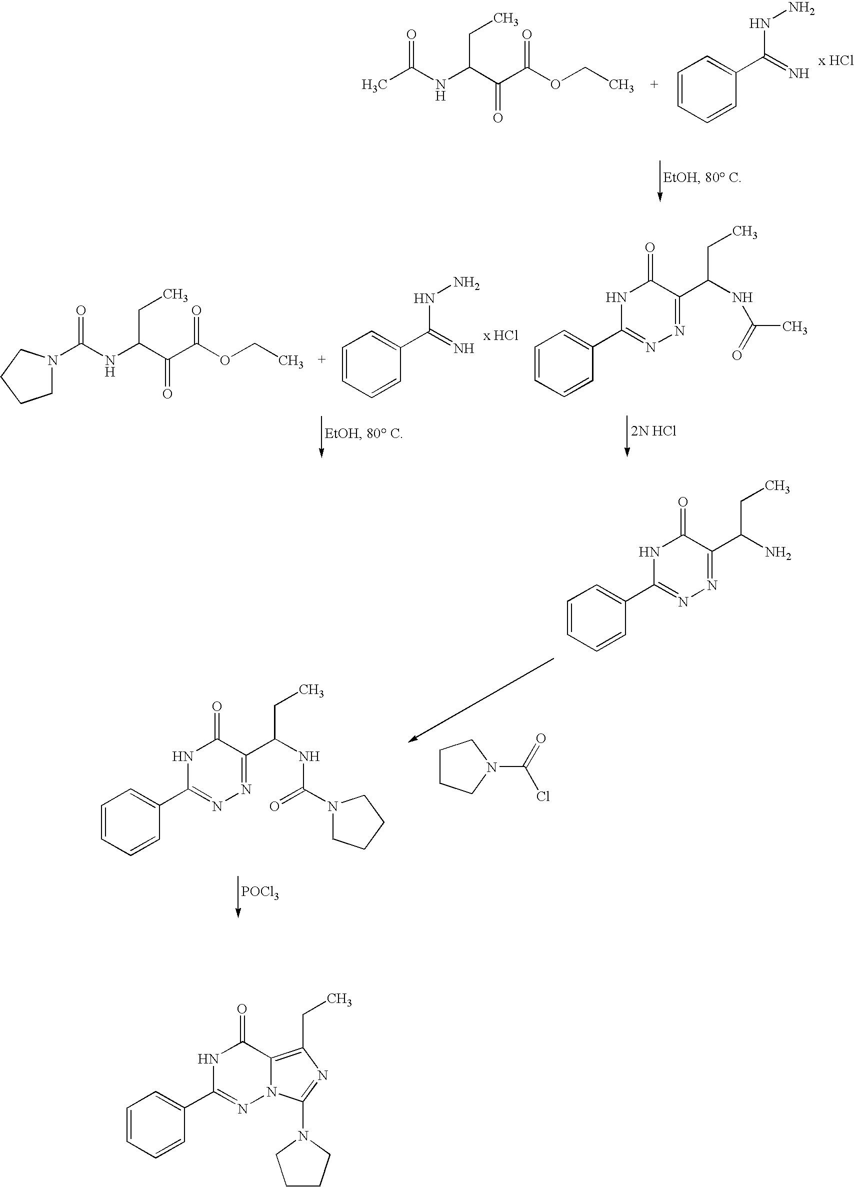

Alternative method for the last steps:

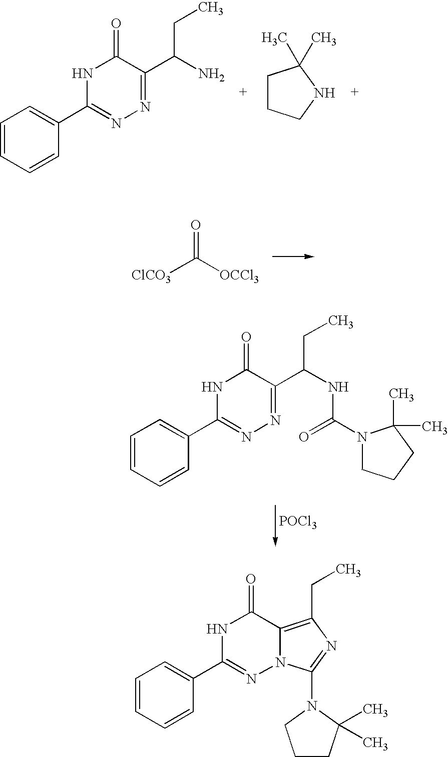

Solvents which are suitable for the individual steps are the customary organic solvents which do not change under the reaction conditions. These preferably include ethers, such as diethyl ether, dioxan, tetrahydrofuran, glycol dimethyl ether, or hydrocarbons, such as benzene, toluene, xylene, hexane, cyclohexane or mineral oil fractions, or halogenated hydrocarbons, such as dichloromethane, trichloromethane, carbon tetrachloride, dichloroethane, trichloroethylene or chlorobenzene, or ethyl acetate, dimethylformamide, dimethylsulfoxide, hexamethylphosphoric triamide, acetonitrile, acetone, or pyridine. It is also possible to use mixtures of the above-mentioned solvents. Particular preference is given to ethanol for the reaction (II)/(IIa)+(II)→(IV)/(IVa), and dichloroethane for the cyclisation (IV)→(I).

The reaction temperature can generally be varied within a relatively wide range. In general, the reaction is carried out in a range of from −20° C. to 200° C., preferably of from 0° C. to 100° C.

The process steps according to the invention are generally carried out under atmospheric pressure. However, it is also possible to operate under superatmospheric pressure or under reduced pressure (for example, in a range from 0.5 to 5 bar).

The compounds of the general formula (IVa) are preferably hydrolysed to compounds of the general formula (V) under acidic conditions as for example in refluxing 2N hydrochloric acid.

The compounds of the general formula (V) are condensed with the compounds of the general formula (VI) to compounds of the general formula (IV) in inert solvents, if appropriate in the presence of a base.

Suitable inert solvents are the customary organic solvents which do not change under the reaction conditions. These preferably include ethers, such as diethyl ether, dioxan, tetrahydrofuran, glycol dimethyl ether, or hydrocarbons, such as benzene, toluene, xylene, hexane, cyclohexane or mineral oil fractions, or halogenated hydrocarbons, such as dichloromethane, trichloromethane, carbon tetrachloride, dichloroethylene, trichloroethylene or chlorobenzene, or ethyl acetate, dimethylformamide, dimethylsulfoxide, hexamethylphosphoric triamide, acetonitrile, acetone, or pyridine. It is also possible to use mixtures of the above-mentioned solvents.

Suitable bases are generally alkali metal hydrides or alkali metal alkoxides, such as, for example, sodium hydride or potassium tert-butoxide, or cyclic amines, such as, for example, piperidine, pyridine, 4-N,N-dimethylaminopyridine or (C₁-C₄)-alkylamines, such as, for example, triethylamine. Preference is given to triethylamine, pyridine and/or 4-N,N-dimethylaminopyridine.

The base is generally employed in an amount of from 1 mol to 4 mol, preferably from 1.2 mol to 3 mol, in each case based on 1 mol of the compound of the formula (V).

The reaction temperature can generally be varied within a relatively wide range. In general, the reaction is carried out in a range of from −20° C. to 200° C., preferably of from 0° C. to 100° C.

Some of the compounds of the general formula (II) are known, or they are novel, and they can then be prepared by converting compounds of the general formula (VI)

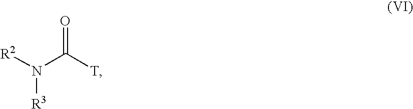

in which

R² and R³ are as defined above, and

T represents a leaving group, preferably chlorine,

initially by reaction with α-aminobutyric acid in inert solvents, if appropriate in the presence of a base and trimethylsilyl chloride, into the compounds of the general formula (VII)

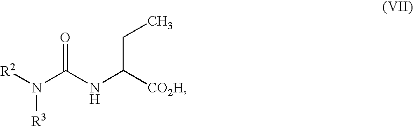

in which

R² and R³ are as defined above,

and finally reacting with the compound of the formula (VII)

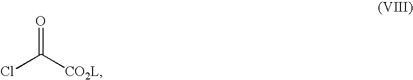

in which

L is as defined above,

in inert solvents, if appropriate in the presence of a base.

The compounds of the general formula (IIa) can be prepared analogously.

Suitable solvents for the individual steps of the process are the customary organic solvents which do not change under the reaction conditions. These preferably include ethers, such as diethyl ether, dioxan, tetrahydrofuran, glycol dimethyl ether, or hydrocarbons, such as benzene, toluene, xylene, hexane, cyclohexane or mineral oil fractions, or halogenated hydrocarbons, such as dichloromethane, trichloromethane, carbon tetrachloride, dichloroethylene, trichloroethylene or chlorobenzene, or ethyl acetate, dimethylformamide, dimethylsulfoxide, hexamethylphosphoric triamide, acetonitrile, acetone, or pyridine. It is also possible to use mixtures of the above-mentioned solvents. Particular preference is given to dichloromethane for the first step and to a mixture of tetrahydrofuran and pyridine for the second step.

Suitable bases are generally alkali metal hydrides or alkali metal alkoxides, such as, for example, sodium hydride or potassium tert-butoxide, or cyclic amines, such as, for example, piperidine, pyridine, 4-N,N-dimethylaminopyridine or (C₁-C₄)-alkylamines, such as, for example, triethylamine. Preference is given to triethylamine, pyridine and/or 4-N,N-dinethylaminopyridine.

The base is generally employed in an amount of from 1 mol to 4 mol, preferably from 1.2 mol to 3 mol, in each case based on 1 mol of the compound of the formula (VII).

The reaction temperature can generally be varied within a relatively wide range. In general, the reaction is carried out in a range of from −20° C. to 200° C., preferably of from 0° C. to 100° C.

The compounds of the general formulae (VI) and (VIII) are known per se, or they can be prepared by customary methods.

The compounds of the general formula (III) are known or can be prepared by reacting compounds of the general formula (IX)
R¹—Y (IX),

in which

R¹ is as defined above, and

Y represents a cyano, carboxyl, methoxycarbonyl or ethoxycarbonyl group,

with ammonium chloride in toluene and in the presence of trimethylaluminium in hexane in a temperature range from −20° C. to room temperature, preferably at 0° C. and atmospheric pressure, and reacting the resulting amidine, if appropriate in situ, with hydrazine hydrate.

The compounds of the general formula (IX) are known per se, or they can be prepared by customary methods.

The compounds of the general formula (I) inhibit the PDE 4 resident in the membranes of human neutrophils. One measured functional consequence of this inhibition was inhibition of superoxide anion production by stimulated human neutrophils.

The compounds of the general formula (I) can therefore be employed in medicaments for the treatment of inflammatory processes, esp. acute and chronic inflammatory processes, and/or immune diseases.

The compounds according to the invention are preferably suitable for the treatment and prevention of inflammatory processes, i.e. acute and chronic inflammatory processes, and/or immune diseases, such as emphysema, alveolitis, shock lung, all kinds of chronic obstructive pulmonary diseases (COPD), adult respiratory distress syndrome (ARDS), asthma, bronchitis, cystic fibrosis, eosinophilic granuloma, arteriosclerosis, arthrosis, inflammation of the gastro-intestinal tract, myocarditis, bone resorption diseases, reperfusion injury, Crohn's disease, ulcerative colitis, systemic lupus erythematosus, type I diabetes mellitus, psoriasis, anaphylactoid purpura nephritis, chronic glomerulonephritis, inflammatory bowel disease, atopic dermatitis, other benign and malignant proliferative skin diseases, allergic rhinitis, allergic conjunctivitis, vernal conjunctivitis, arterial restenosis, sepsis and septic shock, toxic shock syndrome, grafts vs. host reaction, allograft rejection, treatment of cytokine-mediated chronic tissue degeneration, rheumatoid arthritis, arthritis, rheumatoid spondylitis, osteoarthritis, coronary insufficiency, myalgias, multiple sclerosis, malaria, AIDS, cachexia, prevention of tumor growth and tissue invasion, leukemia, depression, memory impairment and acute stroke. The compounds according to the invention are additionally suitable for reducing the damage to infarct tissue after reoxygenation.

The active component can act systemically and/or locally. For this purpose, it can be applied in a suitable manner, for example orally, parenterally, pulmonally, nasally, sublingually, lingually, buccally, rectally, transdermally, conjunctivally, otically or as an implant.

For these application routes, the active component can be administered in suitable application forms.

Useful oral application forms include application forms which release the active component rapidly and/or in modified form, such as for example tablets (non-coated and coated tablets, for example with an enteric coating), capsules, sugar-coated tablets, granules, pellets, powders, emulsions, suspensions, solutions and aerosols.

Parenteral application can be carried out with avoidance of an absorption step (intravenously, intraarterially, intracardially, intraspinally or intralumbarly) or with inclusion of an absorption (intramuscularly, subcutaneously, intracutaneously, percutaneously or intraperitoneally). Useful parenteral application forms include injection and infusion preparations in the form of solutions, suspensions, emulsions, lyophilisates and sterile powders.

Forms suitable for other application routes include for example inhalatory pharmaceutical forms (including powder inhalers, nebulizers), nasal drops/solutions, sprays; tablets or capsules to be administered lingually, sublingually or buccally, suppositories, ear and eye preparations, vaginal capsules, aqueous suspensions (lotions, shake mixtures), lipophilic suspensions, ointments, creams, milk, pastes, dusting powders or implants.

The active components can be converted into the recited application forms in a manner known per se. This is carried out using inert non-toxic, pharmaceutically suitable excipients. These include inter alia carriers (for example microcrystalline cellulose), solvents (for example liquid polyethylene glycols), emulsifiers (for example sodium dodecyl sulphate), dispersing agents (for example polyvinyl-pyrrolidone), synthetic and natural biopolymers (for example albumin), stabilizers (for example antioxidants such as ascorbic acid), colorants (for example inorganic pigments such as iron oxides) or taste and/or odor corrigents.

Generally it has proved advantageous in the case of parenteral application to administer amounts of about 0.001 to 1 mg/kg and preferably about 0.01 to 0.5 mg/kg of body weight to achieve efficacious results. In the case of oral administration, the amount is about 0.001 to 50 mg/kg and preferably about 0.001 to 20 mg/kg of body weight.

In spite of this, it can be necessary in certain circumstances to depart from the amounts mentioned, namely as a function of body weight, application route, individual behaviour towards the active component, manner of preparation and time or interval at which application takes place. It can for instance be sufficient in some cases to use less than the aforementioned minimum amount, while in other cases the upper limit mentioned will have to be exceeded. In the case of the application of larger amounts, it can be advisable to divide them into a plurality of individual doses spread through the day.

The percentages in the tests and examples which follows are, unless otherwise stated, by weight; parts are by weight. Solvent ratios, dilution ratios and concentrations reported for liquid/liquid solutions are each based on the volume.

Test Descriptions

1. Preparation of Human PMN

Human PMN (polymorphonuclear neutrophil leucocytes) are readily purified from peripheral blood. Phosphodiesterase in these cells is predominantly located in the membrane fraction. Inhibitory potency of compounds against this preparation correlate well with the anti-inflammatory activity as measured by inhibiton of superoxide production.

Blood was taken from healthy subjects by venous puncture and neutrophils were purified by dextran sedimentation and density gradient centrifugation on Ficoll Histopaque and resuspended in the buffered medium.

2. Assay of Human PMN Phosphodiesterase

This was performed as a particulate fraction from human PMN essentially as described by Souness and Scott [Biochem. J. 291, 389-395 (1993)]. Particulate fractions were treated with sodium vanadate/glutathione as described by the authors to express the discrete stereospecific site on the phosphodiesterase enzyme. The prototypical PDE 4 inhibitor, rolipram, had an IC₅₀ value in the range 450 nM-1500 nM, thus defining this preparation as the so-called “low affinity” [L] form. The preparation examples had IC₅₀-values within the range of 4 nM -500 nM.

3. Inhibition of FMLP-Stimulated Production of Superoxide Radical Anions

Neutrophils (2.5×10⁵ ml⁻¹) were mixed with cytochrome C (1.2 mg/ml) in the wells of a microtitre plate. Compounds according to the invention were added in dimethyl sulphoxide (DMSO). Compound concentration ranged from 2.5 nM to 10 μM, the DMSO concentration was 0.1% v/v in all wells. After addition of cytochalasin b (5 μg×ml⁻¹) the plate was incubated for 5 min at 37° C. Neutrophils were then stimulated by addition of 4×10⁻⁸ M FMLP (N-Formyl-Met-Leu-Phe) and superoxide generation measured as superoxide dismutase inhibitable reduction of cytochrome C by monitoring the OD₅₅₀ in a Thermomax microtitre plate spectrophotometer. Initial rates were calculated using a Softmax kinetic calculation programme. Blank wells contained 200 units of superoxide dismutase.

The inhibition of superoxide production was calculated as follows:

$$\frac{[ 1 - ( Rx - Rb ) ]}{( Ro - Rb )} \times 100$$

Rx=Rate of the well containing the compound according to the invention

Ro=Rate in the control well

Rb=Rate in the superoxide dismutase containing blank well

4. Assay of Binding to the Rolipram Binding Site (PDE 4 High Affinity Site; “H-PDE 4 form”) in Rat Brain Membranes:

The activity of compounds on the PDE 4 high affinity site (“H-PDE 4 form”) is readily measured by determining their potency for displacement of [³H]-rolipram from its binding site in rat brain membranes. Activity at this site is believed to be a measure of side effect potential (e.g. stimulation of stomach acid secretion, nausea and emesis).

The rolipram binding site assay was performed essentially as described by Schneider et al. [Eur. J. Pharmacol. 127, 105-115 (1986)].

5. Lipopolysaccharide (LPS)—Induced Neutrophil Influx into Rat Lung

Intranasal administration of LPS to rats causes a marked influx of neutrophils into the lungs measurable by histological or biochemical (myeloperoxidase content of the cell pellet) analysis of the bronchoalveolar lavage fluid 24 h later. Rats were treated with test compound or vehicle administered by the oral route 1 h prior to and 6 h after administration of intranasal LPS. 24 hours later animals were euthanatized and their lungs lavaged with PBS (phosphate buffered saline). Neutrophil and total cell numbers were analysed.

6. Emetic Potential in the Marmoset

Vehicle or test compound was administered by the oral route to conscious marmosets. Animals were observed for emetic episodes or abnormal behaviour for 1 h post dosing. In some experiments, if no adverse response was seen, a separate group of animals was tested at ½ log dose higher until emesis or abnormal behaviour was observed. The highest dose at which no abnormal behavior or emetic episodes occurred was recorded as the NOEL.

Materials and Methods

LC-MS method A

| | |
|---|---|
| LC-parameters: | solution A: acetonitrile |
| | solution B: 0.3 g 30% HCl/L water |
| | column oven 50° C.; |
| | column Symmetry C18 2.1 × 150 mm |

| gradient: | time [min] | % A | % B | flow [mL/min] |
|---|---|---|---|---|
| | 0 | 10 | 90 | 0.9 |
| | 3 | 90 | 10 | 1.2 |
| | 6 | 90 | 10 | 1.2 |

LC-MS method B

| | |
|---|---|
| LC-parameters: | solution A: acetonitrile/0.1% formic acid |
| | solution B: water/0.1% formic acid |
| | column oven 40° C.; |
| | column Symmetry C18 2.1 × 50 mm |

| gradient: | time [min] | % A | % B | flow [mL/min] |
|---|---|---|---|---|
| | 0 | 10 | 90 | 0.5 |
| | 4 | 90 | 10 | 0.5 |
| | 6 | 90 | 10 | 0.5 |
| | 6.1 | 10 | 90 | 1.0 |
| | 7.5 | 10 | 90 | 0.5 |

GC-MS method A

| | |
|---|---|
| Column: | HP-5 30 m × 320 μm × 0.25 μm |
| Carrier Gas: | Helium |
| Mode: | Constant flow, initial flow: 1.5 mL/min |
| Oven ramp: | initial temp: 60° C. |
| | initial time: 1 min |
| | rate: 14° C./min up to 300° C., then 300° C. 2 min |

Unless specified otherwise, the following chromatographic conditions were applied: chromatography was performed on silica gel Si 60; for flash chromatography, the usual conditions were followed as described in Still, J. Org. Chem. 43, 2923 (1978); mixtures of dichloromethane and methanol or cyclohexane and ethylacetate were used as eluants. Unless specified otherwise, reactions were executed under an argon atmosphere and under anhydrous conditions.

Abbreviations

HPLC high performance liquid chromatography

MS=mass spectroscopy

NMR=nuclear magnetic resonance spectroscopy

LC-MS=liquid chromatography combined with mass spectroscopy

GC-MS=gas chromatography combined with mass spectroscopy

MeOH=methanol

DMSO=dimethylsulfoxide

THF=tetrahydrofuran

Starting Materials

### EXAMPLE 1A

### 2-(Acetylamino)butanoic acid

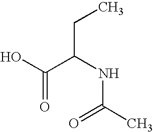

163 g (1.58 mol) 2-Aminobutanoic acid are dissolved in acetic acid, and 242 g (2.37 mol) acetic anhydride are added dropwise. The mixture is stirred for 2 h at 100° C. until completion of reaction, then the solution is evaporated to dryness in vacuo. The solid residue is suspended in ethyl acetate, filtered and washed with diethyl ether.

Yield: 220 g (95.9%)

¹H-NMR (Methanol-d₄): δ/ppm 0.97 (t, 3 H), 1.65-1.93 (m, 2 H), 1.99 (s, 3 H), 4.29 (q, 1 H) ppm.

### EXAMPLE 2A

### Ethyl 3-(acetylamino)-2-oxopentanoate

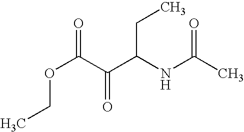

9.2 g (63.4 mmol) 2-(Acetylamino)butanoic acid are suspended in 120 ml tetrahydrofuran and heated to reflux together with 15.0 g (190 mmol) pyridine and a bit of N,N-dimethylaminopyridine. While heating at reflux, 17.3 g (127 mmol) ethyl chloro(oxo)acetate are added dropwise. The reaction mixture is heated at reflux until no more evolution of gas can be observed. After cooling down to room temperature, the reaction mixture is added to ice water and the organic phase extracted with ethyl acetate. The dried organic phase is evaporated to dryness in vacuo, dissolved in ethanol and the solution directly used for the next reaction.

### EXAMPLE 3A

### N-[1-(5-Oxo-3-phenyl-4,5-dihydro-1,2,4-triazin-6-yl)propyl]acetamide

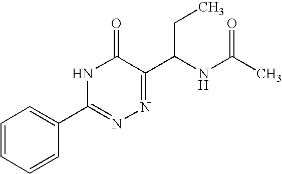

7.26 g (46.8 mmol) Benzenecarboximidamide hydrochloride are suspended in 50 ml of ethanol and 2.54 g (51 mmol) hydrazine hydrate are added. After stirring at room temperature for 1 hour, 13.98 g (69.6 mmol) of the compound of Example 2A, dissolved in 10 ml of ethanol, are added. The reaction mixture is stirred at 80° C. (bath temperature) for 4 hours and then at room temperature over night. The mixture is evaporated to dryness in vacuo and the product is purified by chromatography (flash or column chromatography or preparative HPLC).

Yield: 10.1 g (80%)

¹H-NMR (DMSO-d₆, 300 MHz): δ=0.9 (t, 3H), 1.5 (m, 1H), 1.8 (m, 1H), 1.9 (s, 3H), 4.9 (m, 1H), 7.5 (m, 3H), 8.1 (m, 3H), 14.1 (br. s, 1H) ppm.

### EXAMPLE 4A

### 6-(1-Aminopropyl)-3-phenyl-1,2,4-triazin-5(4H)-one

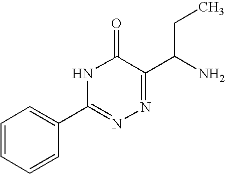

9.00 g (33.0 mmol) of Example 3A are heated to reflux in 150 ml 2.5 N hydrochloric acid for 18 hours. After cooling down to room temperature, the mixture is neutralized with 10% sodium hydroxide and, after addition of ethanol, evaporated to dryness in vacuo. The residue is treated with methanol and the filtrate separated from the salts. The filtrate is evaporated to dryness in vacuo and the product purified by column chromatography.

Yield: 5.9 g (77%)

¹H-NMR (DMSO-d₆, 200 MHz): δ=0.9 (t, 3H), 1.9 (m, 2H), 4.1 (m, 1H), 4.3 (dd, 1H), 7.4 (m, 3H), 8.2 (m., 2H), 8.3 (br. s, 2H) ppm.

### EXAMPLE 5A

### N-[1-(5-Oxo-3-phenyl-4,5-dihydro-1,2,4-triazin-6-yl)propyl]-1-pyrrolidine-carboxamide

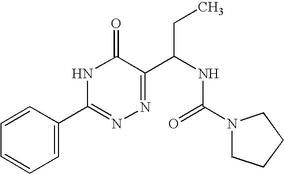

100 mg (0.43 mmol, 1 equiv.) of Example 4A are suspended in 10 ml dichioromethane, 0.08 ml (0.52 mmol, 1.2 equiv.) triethylamine and 64 mg (0.48 mmol, 1.1 equiv.) 1-pyrrolidinecarbonyl chloride are added. The reaction mixture is stirred at room temperature until completion of reaction (1-2 hours). The reaction mixture is added to the same volume of 1 N hydrochloric acid, the organic phase is washed with 1 N hydrochloric acid and brine, dried over sodium sulfate and evaporated to dryness. The product is used without further purification or purified by chromatography (flash or column chromatography or preparative HPLC).

Yield: 140 mg (98%)

LC/MS (A): MS (ES]): 328 (M+H)⁺, retention time 2.74 min.

### EXAMPLE 6A

### N,N-Diethyl-N′-[1-(5-oxo-3-phenyl-4,5-dihydro-1,2,4-triazin-6-yl)propyl]urea

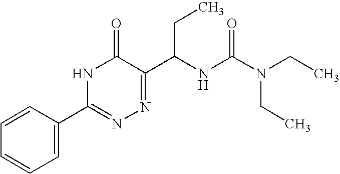

In analogy to the procedure for Example 5A, 100 mg (0.43 mmol) of Example 4A, 65 mg (0.48 mmol) diethylcarbamic chloride and proportionate amounts of the other reagents are used.

Yield: 140 mg (98%)

LC/MS (A): MS (ESI): 330 (M+H)⁺, retention time 2.96 min.

### EXAMPLE 7A

### N-[1-(5-Oxo-3-phenyl-4,5-dihydro-1,2,4-triazin-6-yl)propyl]-4-morpholine-carboxamide

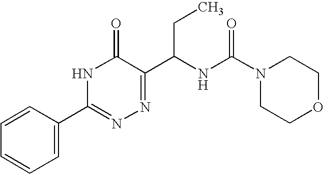

In analogy to the procedure for Example 5A, 100 mg (0.43 mmol) of Example 4A, 71 mg (0.48 mmol) 4-morpholinecarbonyl chloride and proportionate amounts of the other reagents are used.

Yield: 150 mg (99%)

LC/MS (A): MS (ESI): 344 (M+H)⁺, retention time 2.39 min.

### EXAMPLE 8A

### M-[1-(5-Oxo-3-phenyl-4,5-dihydro-1,2,4-triazin-6-yl)propyl]-N,N-dipropylurea

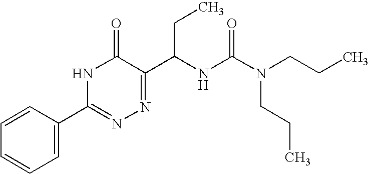

In analogy to the procedure for Example 5A, 100 mg (0.43 mmol) of Example 4A, 71 mg (0.48 mmol) dipropylcarbarmic chloride and proportionate amounts of the other reagents are used.

Yield: 140 mg (90%)

LC/MS (A): MS (ESI): 358 (M+H)⁺, retention time 3.47 min.

### EXAMPLE 9A

### 5N-[1-(5-Oxo-3-phenyl-4,5-dihydro-1,2,4-triazin-6-yl)propyl]-3,4-dihydro-1(2H-quinolinecarboxamide

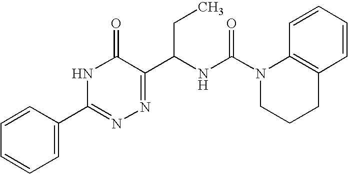

In analogy to the procedure for Example 5A, 100 mg (0.43 mmol) of Example 4A, 93 mg (0.48 mmol) 3,4-dihydro-1(2H)-quinolinecarbonyl chloride and proportionate amounts of the other reagents are used.

Yield: 150 mg (88%)

LC/MS (A): MS (ESI): 390 (M+H)⁺, retention time 3.53 min.

### EXAMPLE 10A

### N-[1-(5-Oxo-3-phenyl-4,5-dihydro-1,2,4-triazin-6-yl)propyl]-3-azabicyclo[3.2.0]-heptane-3-carboxamide

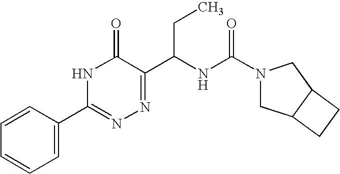

In analogy to the procedure for Example 5A, 100 mg (0.43 mmol) of Example 4A, 76 mg (0.48 mmol) 3-azabicyclo[3.2.0]heptane-3-carbonyl chloride and proportionate amounts of the other reagents are used.

Yield: 140 mg (91%)

LC/MS (A): MS (ESI): 354 (M+H)⁺, retention time 3.11 min.

### EXAMPLE 11A

### 4-Methyl-N-[1-(5-oxo-3-phenyl-4,5-dihydro-1,2,4-triazin-6-yl)propyl)-1-piperazine-carboxamide

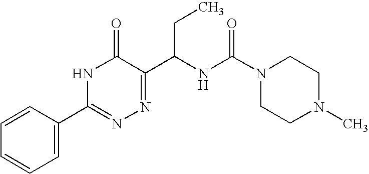

In analogy to the procedure for Example 5A, 100 mg (0.43 mmol) of Example 4A, 95 mg (0.48 mmol) 4-methylpiperazinecarbonyl chloride and proportionate amounts of the other reagents are used.

Yield: 150 mg (99%)

LC/MS (A): MS (ESI): 357 (M+H)⁺, retention time 0.31 min.

### EXAMPLE 12A

### 4-tert-Butyl-N-[1-(5-oxo-3-phenyl-4,5-dihydro-1,2,4-triazin-6-yl)propyl]-1-piperidinecarboxamide

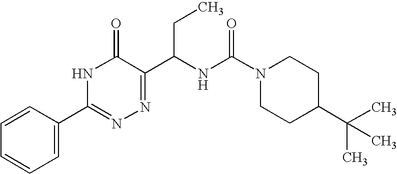

50 mg (0.17 mmol, 0.33 equiv.) Triphosgene are dissolved under argon in 5 ml THF. The solution is cooled to 0° C. and a solution of 72 mg (0.51 mmol, 1 equiv.) 4-tertbutylpiperidine in THF is added dropwise. The mixture is stirred for 5 minutes and 0.21 ml (1.53 mmol, 3 equiv.) triethylamine are added dropwise. The mixture is warmed to room temperature, stirred for 15 minutes and cooled again to 0° C. 118 mg (0.51 mmol, 1 equiv.) of Example 4A are added and the reaction mixture is stirred for 1 hour at 0° C. and overnight at room temperature. The reaction mixture is diluted with dichloromethane and filtered, the organic phase is washed once with water, dried over magnesium sulfate and evaporated to dryness. The product is used without further purification or purified by chromatography (flash or column chromatography or preparative HPLC).

Yield: 150 mg (99%)

LC/MS (A): MS (ESI): 398 (M+H)⁺, retention time 3.95 min.

### EXAMPLE 13A

### N-[1-(5-Oxo-3-phenyl-4,5-dihydro-1,2,4-triazin-6-yl)propyl]-3-azabicyclo[3.2.1]-octane-3-carboxamide

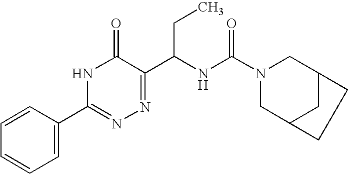

In analogy to the procedure for Example 12A, 160 mg (0.69 mmol) of Example 4A, 77 mg (0.69 mmol) 3-azabicyclo[3.2.1]octane and proportionate amounts of the other reagents are used.

Yield: 200 mg (78%)

LC/MS (A): MS (ESI): 368 (M+H)⁺, retention time 3.31 min.

### EXAMPLE 14A

### 2-Ethyl-N-[1-(5-oxo-3-phenyl-4,5-dihydro-1,2,4-triazin-6-yl)propyl]-1-piperidine-carboxamide

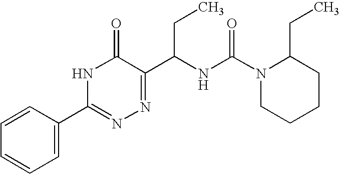

In analogy to the procedure for Example 12A, 136 mg (0.59 mmol) of Example 4A, 67 mg (0.59 mmol) 2-ethylpiperidine and proportionate amounts of the other reagents are used.

Yield: 200 mg (91%)

LC/MS (A): MS (ESI): 329 (M+H)⁺, retention time 3.31 min.

### EXAMPLE 15A

### 4-Ethyl-N-[1-(5-oxo-3-phenyl-4,5-dihydro-1,2,4-triazin-6-yl)propyl]-1-piperidine-carboxamide

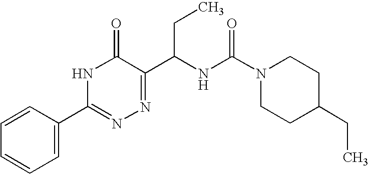

In analogy to the procedure for Example 12A, 183 mg (0.80 mmol) of Example 4A, 90 mg (0.80 mmol) 4-ethylpiperidine and proportionate amounts of the other reagents are used.

Yield: 200 mg (91%)

LC/MS (A): MS (ESI): 370 (M+H)⁺, retention time 3.61 min.

### EXAMPLE 16A

### 4,4-Dimethyl-N-[1-(5-oxo-3-phenyl-4,5-dihydro-1,2,4-triazin-6-yl)propyl]-1-piperidinecarboxamide

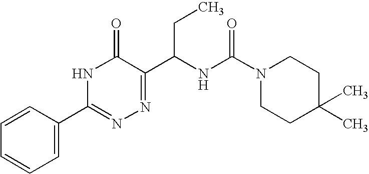

In analogy to the procedure for Example 12A, 200 mg (0.87 mmol) of Example 4A, 98 mg (0.87 mmol) 4,4-dimethylpiperidine and proportionate amounts of the other reagents are used.

Yield: 280 mg (87%)

LC/MS (A): MS (ESI): 370 (M+H)⁺, retention time 3.50 min.

### EXAMPLE 17A

### 2,2-Dimethyl-N-[1-(5-oxo-3-phenyl-4,5-dihydro-1,2,4-triazin-6-yl)propyl]-1-pyrrolidinecarboxamide

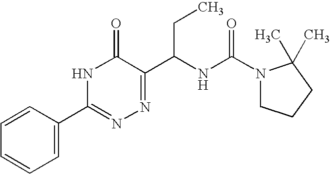

In analogy to the procedure for Example 12A, 150 mg (0.65 mmol) of Example 4A, 89 mg (0.65 mmol) 2,2-dimethylpyrrolidine hydrochloride and proportionate amounts of the other reagents are used.

Yield: 200 mg (86%)

LC/MS (A): MS (ESI): 356 (M+H)⁺, retention time 3.29 min.

### EXAMPLE 18A

### 2-Methyl-N-[1-(5-oxo-3-phenyl-4,5-dihydro-1,2,4-triazin-6-yl)propyl]-1-piperidine-carboxamide

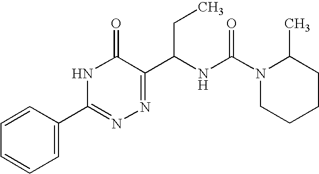

In analogy to the procedure for Example 12A, 151 mg (0.65 mmol) of Example 4A, 65 mg (0.65 mmol) 2-methylpiperidine and proportionate amounts of the other reagents are used.

Yield: 215 mg (92%)

LC/MS (A): MS (ESI): 356 (M+H)⁺, retention time 3.25 min.

### PREPARATION EXAMPLES

### EXAMPLE 1

### 5-Ethyl-2-phenyl-7-(1-pyrrolidinyl)imidazo[5,1-f][1,2,4]triazin-4(3H)-one

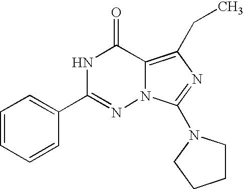

150 mg (0.46 mmol, 1 equiv.) of Example 5A are suspended in 10 ml dichloroethane, and 247 mg (1.6 mmol, 3 equiv.) phosphoroxychloride are added. The mixture is stirred at reflux for 2 hours. After cooling down to room temperature, the solution is evaporated to dryness in vacuo. The product is purified by chromatography (flash or column chromatography or preparative HPLC).

Yield: 32 mg (23%)

LC/MS (A): MS (ES]): 310 (M+H)⁺, retention time 2.67 min.

### EXAMPLE 2

### 7-(Diethylamino)-5-ethyl-2-phenylimidazo[5,1-f][1,2,4]triazin-4(3H)-one

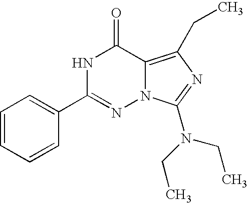

In analogy to the procedure for Example 1, 150 mg (0.45 mmol) of Example 6A, 247 mg (1.61 mmol) phosphoric trichloride are stirred at reflux for 2 hours, proportionate amounts of the solvents are used.

Yield: 29 mg (20%)

LC/MS (A): MS (ESI): 312 (M+H)⁺, retention time 2.99 min.

### EXAMPLE 3

### 5-Ethyl-7-(4-morpholinyl)-2-phenylimidazo[5,1-f][1,2,4]triazin-4(3H)-one

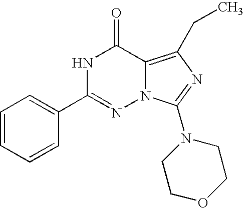

In analogy to the procedure for Example 1, 150 mg (0.44 mmol) of Example 7A, 247 mg (1.61 mmol) phosphoric trichloride are stirred at reflux for 2 hours, proportionate amounts of the solvents are used.

Yield: 27 mg (19%)

LC/MS (A): MS (ESI): 326 (M+H)⁺, retention time 3.02 min.

### EXAMPLE 4

### 7-(Dipropylamino)-5-ethyl-2-phenylimidazo[5,1-f[1,2,4]triazin-4(3H)-one

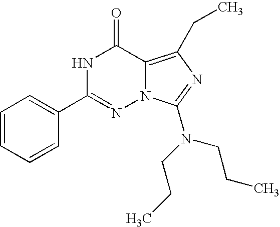

In analogy to the procedure for Example 1, 140 mg (0.39 mmol) of Example 8A, 165 mg (1.07 mmol) phosphoric trichloride are stirred at reflux for 2 hours, proportionate amounts of the solvents are used.

Yield: 12 mg (9%)

¹H-NMR (DMSO, 300 MHz): δ=0.90 (m, 9H); 1.20 (m, 4H); 1.62 (m, 41); 2.80 (quart., 2H); 3.50 (m, 4H); 7.55 (m, 3H); 7.90 (m, 2H); 11.50 (s, 1H) ppm.

### EXAMPLE 5

### 7-(3,4-Dihydro-1(2H)-quinolinyl)-5-ethyl-2-phenlylimidazo[5,1-f[1,2,4]triazin-4(3H)-one

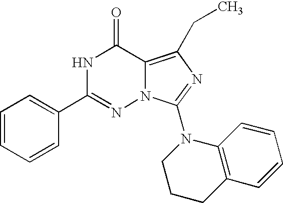

In analogy to the procedure for Example 1, 150 mg (0.39 mmol) of Example 9A, 165 mg (1.07 mmol) phosphoric trichloride are stirred at reflux for 2 hours, proportionate amounts of the solvents are used.

Yield: 11 mg (8%)

¹H-NMR (DMSO, 300 MHz): δ=1.25 (t, 3H); 2.00 (m, 2H); 2.85 (m, 2H); 2.90 (quart., 2H); 3.80 (m, 2H); 6.52 (m, 1H); 6.75 (m, 1H); 6.95 (m, 1H); 7.07 (m, 1H); 7.55 (m, 3H); 7.80 (m, 2H); 11.80 (s, 1H) ppm.

### EXAMPLE 6

### 7-(3-Azabicyclo[3.2.0]hept-3-yl)-5-ethyl-2-phenylimidazo[5,1-f][1,2,4]triazin-4(3H)-one

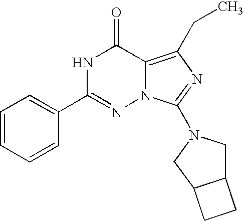

In analogy to the procedure for Example 1, 140 mg (0.39 mmol) of Example 10A, 165 mg (1.07 mmol) phosphoric trichloride are stirred at reflux for 2 hours, proportionate amounts of the solvents are used.

Yield: 69 mg (52%)

¹H-NMR (DMSO, 300 MHz): δ=1.20 (t, 3H); 1.70 (m, 2H); 2.20 (m, 2H); 2.90 (quart., 2H); 3.00 (m, 2H); 3.40 (m, 2H); 4.07 (d, 2H); 7.54 (m, 3H); 7.90 (m, 2H); 11.70 (s, 1H) ppm.

### EXAMPLE 7

### 5-Ethyl-7-(4-methyl-1-piperazinyl)-2-phenylimidazo[5,1-f][1,2,4]triazin-4(3H)-one

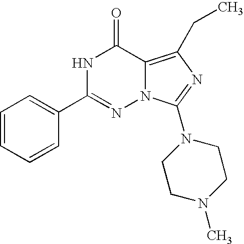

In analogy to the procedure for Example 1, 160 mg (0.45 mmol) of Example 11A, 165 mg (1.07 mmol) phosphoric trichloride are stirred at reflux for 2 hours, proportionate amounts of the solvents are used.

Yield: 6 mg (4%)

¹H-NMR (DMSO, 300 MHz): δ=1.20 (t, 3H); 2.90 (m, 5H); 3.30 (m, 4H); 3.50 (m, 2H); 4.30 (d, 2H); 7.54 (m, 3H); 7.95 (m, 2H); 11.70 (s, 1H) ppm.

### EXAMPLE 8

### 7-(4-tert-Butyl-1-piperidinyl)-5-ethyl-2-phenylimidazo[5,1-f][1,2,4]triazin-4(3H)-one

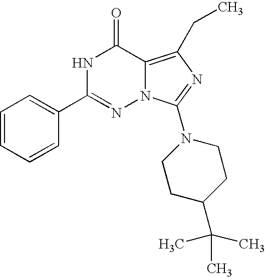

In analogy to the procedure for Example 1, 150 mg (0.45 mmol) of Example 12A, 165 mg (1.07 mmol) phosphoric trichloride are stirred at reflux for 2 hours, proportionate amounts of the solvents are used.

Yield: 72 mg (50%)

¹H-NMR (DMSO, 300 MHz): δ=0.80 (s, 9H); 1.00-1.50 (m, 8H); 1.70 (m, 2H); 2.90 (m, 2H); 4.30 (d, 2H); 7.50 (m, 3H); 7.95 (m, 2H); 11.80 (s, 1H) ppm.

### EXAMPLE 9

### 7-(3-Azabicyclo[3.2.1]oct-3-yl)-5-ethyl-2-phenylimidazo[5,1-f][1,2,4]triazin-4(3H)-one

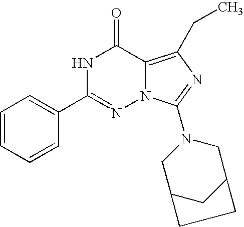

In analogy to the procedure for Example 1, 200 mg (0.54 mmol) of Example 13A, 100 mg (0.65 mmol) phosphoric trichloride are stirred at reflux for 2 hours, proportionate amounts of the solvents are used.

Yield: 190 mg (99%)

¹H-NMR (DMSO, 300 MHz): δ=1.20 (t, 3H); 1.50-3.00 (m, 12H); 4.01 (d, 2H); 7.62 (m, 3H); 7.96 (m, 2H); 11.64 (s, 1H) ppm.

### EXAMPLE 10

### 5-Ethyl-7-(2-ethyl-1-piperidinyl)-2-phenylimidazo[5,1-f][1,2,4]triazin4(3H)-one

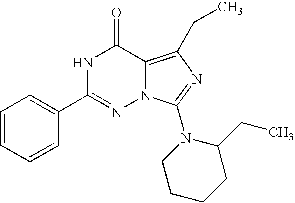

In analogy to the procedure for Example 1, 200 mg (0.54 mmol) of Example 14A, 100 mg (0.65 mmol) phosphoric trichloride are stirred at reflux for 2 hours, proportionate amounts of the solvents are used.

Yield: 185 mg (97%)

¹H-NMR (DMSO, 300 Mz): δ=0.80 (t, 3H); 1.00 (t, 3H); 1.50-2.00 (m, 6H); 2.90 (m, 2H); 3.20 (m, 2H); 3.35 (m, 1H); 4.05 (m, 1H); 4.50 (m, 1H); 7.65 (m, 3H); 7.98 (m, 2H); 11.80 (s, 1H) ppm.

### EXAMPLE 11

### 5-Ethyl-7-(4-ethyl-1-piperidinyl)-2-phenylimidazo[5,1-f][1,2,4]triazin-4(3H)-one

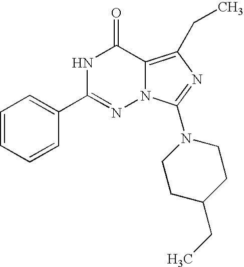

In analogy to the procedure for Example 1, 200 mg (0.54 mmol) of Example 15A, 250 mg (1.61 mmol) phosphoric trichloride are stirred at reflux for 2 hours, proportionate amounts of the solvents are used.

Yield: 33 mg (17%)

¹H-NMR (DMSO, 300 MHz): δ=0.90 (t, 3H); 1.15 (t, 3H); 1.30 (m, 5H); 1.75 (m, 2H); 2.90 (m, 4H); 4.20 (m, 2H); 7.50 (m, 3H); 7.90 (m, 2H); 11.44 (s, 1H) ppm.

### EXAMPLE 12

### 5-Ethyl-7-(2,2-dimethyl-1-piperidinyl)-2-phenylimidazo[5,1-f][1,2,4]triazin-4(3H)-one

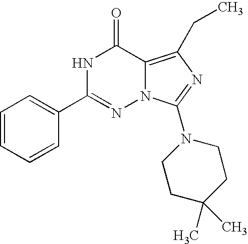

In analogy to the procedure for Example 1, 280 mg (0.76 mmol) of Example 16A, 247 mg (1.61 mmol) phosphoric trichloride are stirred at reflux for 2 hours, proportionate amounts of the solvents are used.

Yield: 38 mg (14%)

¹H-NMR (DMSO, 300 MHz): δ=0.90 (s, 6H); 1.20 (t, 3H); 1.47 (m, 4H); 1.70 (m, 1H); 2.08 (m, 1H); 2.90 (m, 2H); 3.60 (m, 2H); 7.50 (m, 3H); 7.90 (m, 2H); 11.80 (s, 1H) ppm.

### EXAMPLE 13

### 7-(2,2-Dimethyl-1-pyrrolidinyl)-5-ethyl-2-phenylimidazo[5,1-f][1,2,4]triazin-4(3H)-one

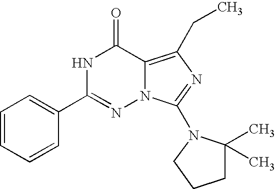

In analogy to the procedure for Example 1, 200 mg (0.56 mmol) of Example 17A, 247 mg (1.61 mmol) phosphoric trichloride are stirred at reflux for 2 hours, proportionate amounts of the solvents are used.

Yield: 12 mg (7%)

¹H-NMR (DMSO, 300 MHz): δ=1.20 (t, 3H); 1.50 (s, 6H); 1.70 (m, 2H); 1.90 (m, 2H); 2.80 (q, 2H); 4.00 (m, 2H); 7.50 (m, 31H); 7.90 (m, 2H); 11.20 (s, 1H) ppm

### EXAMPLE 14

### 5-Ethyl-7-(2-methyl-1-piperidinyl)-2-phenylimidazo[5,1-f][1,2,4]triazin-4(3H)-one

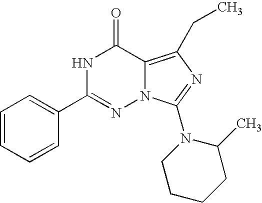

In analogy to the procedure for Example 1, 215 mg (0.60 mmol) of Example 18A, 247 mg (1.65 mmol) phosphoric trichloride are stirred at reflux for 2 hours, proportionate amounts of the solvents are used.

Yield: 19 mg (10%)

¹H-NMR (DMSO, 300 MHz): δ=1.20 (m, 6H); 1.40-2.00 (m, 6H); 2.90 (m, 2H); 3.35 (m, 1H); 3.75 (m, 1H); 4.50 (m, 1H); 7.55 (m, 3H); 7.90 (m, 2H); 11.50 (s, 1H) ppm.

We claim:

1. Compounds of the general formula (I) 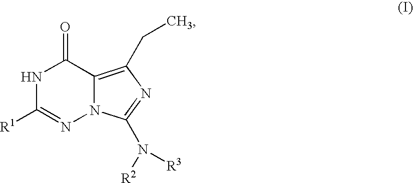
in which
R¹ denotes phenyl or 5- to 6-membered heteroaryl, which can be substituted by 0, 1, 2 or 3 residues independently selected from the group consisting of halogen, (C₁-C₄)-alkyl, (C₁-C₄)-alkoxy, trifluoromethyl, cyano, nitro and trifluoromethoxy,
R² denotes hydrogen, (C₁-C₈)-alkyl or (C₃-C₈)-cycloalkyl, and
R³ denotes (C₁-C₈)-alkyl or (C₃-C₈)-cycloalkyl, or
NR²R³ denotes optionally benzannelated, 4- to 10-membered heterocyclyl,
which contains at least one nitrogen ring atom,
which is attached to the imidazotriazinone by a nitrogen ring atom, and
which can be substituted by 0, 1, 2 or 3 residues independently selected from the group consisting of halogen, (C₁-C₆)-alkyl, (C₁-C₆)-alkoxy, (C₁-C₆)-alkoxycarbonyl, hydroxy, halogen, trifluoromethyl and oxo.

2. Compounds according to claim 1, whereby
R¹ denotes phenyl, which can be substituted by 0, 1, 2 or 3 residues independently selected from the group consisting of fluoro, chloro, methyl, ethyl, trifluoromethyl and cyano, and
R²and R³ have the meaning indicated in claim 1.

3. Compounds according to claim 1, whereby
R¹ denotes phenyl, which can be substituted by 0, 1, 2 or 3 residues independently selected from the group consisting of fluoro, chloro, methyl, ethyl, trifluoromethyl and cyano, and
R² and R³ are identical or different and denote (C₁-C₆)-alkyl or (C₃-C₈)-cyclo-alkyl, or
NR²R³ denotes optionally benzannelated, 4- to 10-membered heterocyclyl,
which contains at least one nitrogen ring atom,
which is attached to the imidazotriazinone by a nitrogen ring atom, and
which can be substituted by 0, 1, 2 or 3 residues independently selected from the group consisting of (C₁-C₄)-alkyl, (C₁-C₄)-alkoxy, and (C₁-C₄)-alkoxycarbonyl.

4. A process for the preparation of the compounds according to claim 1, characterized in that
compounds of the general formula (IV) 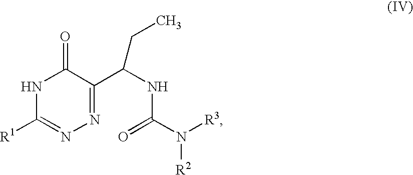
in which R¹, R² and R³ have tile meaning indicated in claim 1,
are reacted with a dehydrating agent.

5. Compounds of the general formula (IV) according to claim 4.

6. Compounds according to any one of claims 1 to 3 for therapeutic and/or prophylactic use.

7. Pharmaceutical composition containing at least one compound according to any one of claims 1 to 3 and a pharmacologically acceptable diluent.

8. Use of compounds according to any one of claims 1 to 3 for the preparation of medicaments.

9. Use of compounds according to any one of claims 1 to 3 for the preparation of medicaments for the treatment and/or prophylaxis of inflammatory processes and/or immune diseases.

10. Use of compounds according to any one of claims 1 to 3 for the preparation of medicaments for the treatment and/or prophylaxis of chronic obstructive pulmonary disease and/or asthma.

11. Process for controlling asthma or chronic obstructive pulmonary disease in humans and animals by administration of an antiinflammatory effective amount of at least one compound according to any of claims 1 to 3.
